(19) United States
(12) Patent Application Publication
(10) Pub. No.: US 2025/0157599 A1
(43) Pub. Date: May 15, 2025

(54) ARTIFICIAL INTELLIGENCE AIDED IDENTIFICATION OF PARTICIPANTS FOR CLINICAL TRIALS AND PRECISION MEDICINE

(71) Applicant: Medzown, Inc., Jupiter, FL (US)

(72) Inventors: Jennifer Levin Carter, Palm Beach Gardens, FL (US); Nancy Lyn Jensen Biery, Kenmore, WA (US); Raul Hugo Curbelo, Sturbridge, MA (US)

(73) Assignee: Medzown, Inc., Jupiter, FL (US)

(21) Appl. No.: 18/949,428

(22) Filed: Nov. 15, 2024

Related U.S. Application Data
(60) Provisional application No. 63/599,391, filed on Nov. 15, 2023.

(51) Int. Cl. G16H 10/20; G06Q 30/0283; G16H 40/67; G16H 50/30; G16H 50/70

(52) U.S. Cl. CPC G16H 10/20; G06Q 30/0283; G16H 40/67; G16H 50/30; G16H 50/70

(57) ABSTRACT

Automated systems with controllers having a processor and a digital storage that may be operative as an artificial intelligence engine capable of receiving digital data descriptive of physiological metrics objectively quantifying one or more physical attributes of patients and receiving in digital data having a subjective health assessment of the patients. The controller processes the input data with potential physiological effects of a health treatment to a participant included in a clinical trial, and a length of time that the participant included in the clinical trial will need to receive treatment, to generate an assessment of whether the patient will benefit from inclusion as a participant in the clinical trial.

### CROSS REFERENCE TO RELATED APPLICATIONS

This application claims priority to U.S. Provisional Application No. 63/599,391, filed Nov. 15, 2023, and entitled ARTIFICIAL INTELLIGENCE AIDED IDENTIFICATION OF PARTICIPANTS IN CLINICAL TRIALS AND PRECISION MEDICINE, the entire disclosure of which is incorporated by reference herein.

### FIELD OF THE DISCLOSURE

The present disclosure relates to methods and apparatus for improved medicine management using artificial intelligence. More specifically, the present invention provides methods and apparatus to align patients, health care providers, payers, and therapeutic agent developers to facilitate access to advanced treatments by patients with complex diseases.

### BACKGROUND OF THE DISCLOSURE

It is estimated that five percent (5%) of the population consume fifty percent (50%) or more of the cost of care available to a society (historically, 80% of healthcare spend has been associated with the sickest 20% of all patients). Consequently, more than One Trillion Dollars (USD) is projected to be spent solely on rare diseases in upcoming years. Nearly 65% of new Food and Drug Administration (FDA) approvals since 2019 have been for specialty drugs. Such specialty drug costs are quickly growing and are expected to increase from 17% of all FDA approved pharmaceuticals to 24% in upcoming years.

While such breakthrough advanced therapies are promising to patients, the costs of specialty drugs are rapidly escalating. Such expenditures compete with general wellness objectives for the general population, and do not take into account the ancillary impacts and costs to society of rare and complex illnesses. Piecemeal rebates, coupons, and similar programs become cost-shifting mechanisms and do not holistically improve costs or patient outcomes.

Clinical trials are an important part of accepted practices for bringing new treatments to the public. However, current practices for finding suitable participants for specific clinical trials are time consuming and lack consistency. Often qualified participants are not identified for a given clinical, or by the time that they are identified, their disease state has progressed to a stage indicating that the participant is no longer a good match for the clinical.

The current systems for developing and bringing targeted therapies to the public in affordable, timely, and effective ways need to become more powerful and efficient.

### SUMMARY OF THE DISCLOSURE

The present invention provides methods and apparatus for improved functions, performance and overcoming of shortcomings of currently implemented medicine management systems.

The present invention allows for objective physiological data descriptive of a patient's biological state and subjective physiological data capturing a patient's health experience are received are input into an automated controller system. In some preferred embodiments, objective and/or subjective physiological data are received at multiple time points on a time continuum.

Physiological effects anticipated to be experienced as a result of treatment during a clinical trial are also input into the controller system, as well as patient non-physiological status and clinical non-physiological status. The controller will process the received data and prove output indicative of whether one or more patients are good candidates for inclusion as participants in the clinical trial.

Disparate bodies of data compiled during multiple stages of therapeutic agent development are coordinated and interpreted to expedite the identification of therapeutic agents and therapy protocols as they relate to specific patient needs.

Apparatus combines multiple processor designs and logic to assess patient physiological aspects and metrics, and provide statistical input regarding a likelihood of efficacious results from available and/or proposed treatment protocols, including protocols in trial stages or proposed for trials. Automated processes timely provide patients with treatment options, and clinical trial managers with suitable clinical trial participants, using one or more of: Artificial Intelligence (“AI”), structured queries, unstructured queries, Type 1 processing (fast, affect driven, intuitive processes), Type 2 processing (deliberative, logic based processes), image processing (including AI analysis of pixel patterns and polygons derived from static image data generated along a time continuum), statistical analysis, geographic location determination, transportation modalities, and physiological sensors.

The present invention executes advanced data assimilation processes to quickly ingest and organize data from disparate sources. Specialized processors use machine learning and statistical analysis to correlate data descriptive of a patient's health condition, as well as the patient's geographical location, physiological state, and available funding, with the organized ingested data to generate suggested remedial actions. The suggested remedial actions may be accompanied with a statistical projection of an efficacious outcome of one or more remedial actions.

Remedial actions may include precision therapeutics and clinical trial strategies. Benefits of precision therapeutics and clinical trial strategies may include increased control over a disease state and reduced side effects caused by aggressive use of non-targeted medicines. Additional benefits include reduction in ineffective expenditures; faster identification of appropriate clinical trial patients; and more efficient use of existing medical infrastructure.

The methods and apparatus described herein streamline patient and provider access to data-driven, advanced treatment strategies, we align payer, employer, provider, and pharma incentives and address the rising cost of specialty drugs and high-cost claimants.

According to the present invention, machine learning and statistical analysis qualify a patient for “pre-authorization” for one or multiple clinical trials. By taking into consideration volumes and types of data that are not humanly possible to assimilate and apply to a given situation, as well as patient and/or practitioner preferences, the methods and apparatus presented herein are capable of generating weighted choices for one or more potential healthcare strategies, including a roadmap indicating whether a particular course of healthcare treatment in the near future may enhance, obviate, or preclude a different subsequent healthcare option.

Machines capable of working around the clock, every day of the year, and located in environments that are not subjected to sanitizing requirements of healthcare facilities augment the efforts of healthcare teams that are often overworked, in crowded environments, and require significant resources to deploy. The present invention also provides a level of consistency and best practice implementation simply not possible with teams of transient medical professionals.

Other technical features may be readily apparent to one skilled in the art from the following figures, descriptions, and claims.

### BRIEF DESCRIPTION OF THE DRAWINGS

FIG. 1 illustrates a schematic diagram of illustrates sources of disparate data that may be aggregated in an AI engine and stored in data storage.

FIG. 1A illustrates a schematic diagram and description for an AI driven strategy for personalized clinical trial matching is illustrated.

FIG. 2 illustrates aspects that may be addressed via the AI assisted personalized clinical trial matching system is illustrated.

FIG. 3 illustrates aspects of a process that may be implemented by an AI Engine driven prioritization of potential participants for clinical trials.

FIG. 4 illustrates a schematic diagram of a treatment distribution model that may be used to promote health equity goals.

FIG. 5 illustrates exemplary adverse health conditions and patient counts that may be processed by an AI Engine driven mechanism.

FIG. 6 illustrates a schematic diagram of case studies of exemplary adverse health conditions.

FIG. 7 illustrates a schematic diagram of automated assistance in compliance with the regulation guidance.

FIG. 8 illustrates a schematic diagram of automated assistance for patient centric engagement for beneficial health treatment outcomes.

FIG. 9 illustrates a schematic diagram of exemplary for AI guidance for clinical trials.

FIG. 10 illustrates a schematic diagram of aspects included in an automation assisted treatment participation.

FIG. 11 illustrates factors involved in automated weighting of physiological and non-physiological aspects to provide a scaled indicator of successful inclusion in a healthcare treatment.

FIG. 12 illustrates exemplary apparatus that may be used to implement aspects of the present invention including executable software.

FIG. 13 illustrates exemplary controller architecture for use with the present disclosure.

FIGS. 14A-14C illustrate exemplary method steps that may be executed according to some embodiments of the present invention.

### DETAILED DESCRIPTION

According to the present invention, data of various types are conveyed in disparate modalities is received into an AI engine. The AI engine may use raw data, manipulated data, interpreted data, new data and data types generated from existing data. Data may include one or more of: text, image, numerical, pixel patterns, polygons, vectors, molecular, neural, digital, and analog data modalities. In some embodiments, a data input may be received in a first format and converted to another format to aid in analyzation and AI processes.

Data sources may include, by way of non-limiting example, one or more of: a patient portal; a healthcare provider; pharmaceutical developers, government, health insurance companies or other payers, diagnostic labs, or other source.

As described more fully in the following sections, improvements in apparatus and methodology are provided which address observed deficiencies and operational failure relating to: methods used for medical clinical trials, for specialized healthcare, and for treatment of rare and complex medical conditions. Specific examples and embodiments of the improvements are defined herein, however, alternatives and modifications of the provided examples that are consistent with the claimed innovations are within the scope of the present disclosure.

AI engine processing may include one more of: converting image data to pixel patterns and/or polygon patterns, manipulating pixel patterns and/or polygon patterns, analyzing pixel patterns and/or polygon patterns, optical character recognition, alphanumeric analysis, symbol recognition and the like. Proposed treatment strategies, protocols and opportunities may be associated with a diagnosis or with a diagnosis of an associated disease state.

The present invention provides for the deployment of computational frameworks combining disparate aspects of technology to perform tasks that are beyond the ability of traditional healthcare systems or human intelligence. These systems aggregate large volumes of disparate data that may or may not be intuitively linked to healthcare, and utilize multiple modalities data manipulation, algorithms, and statistical models to generate proposed healthcare strategies for a patient (or group of similarly situated patients).

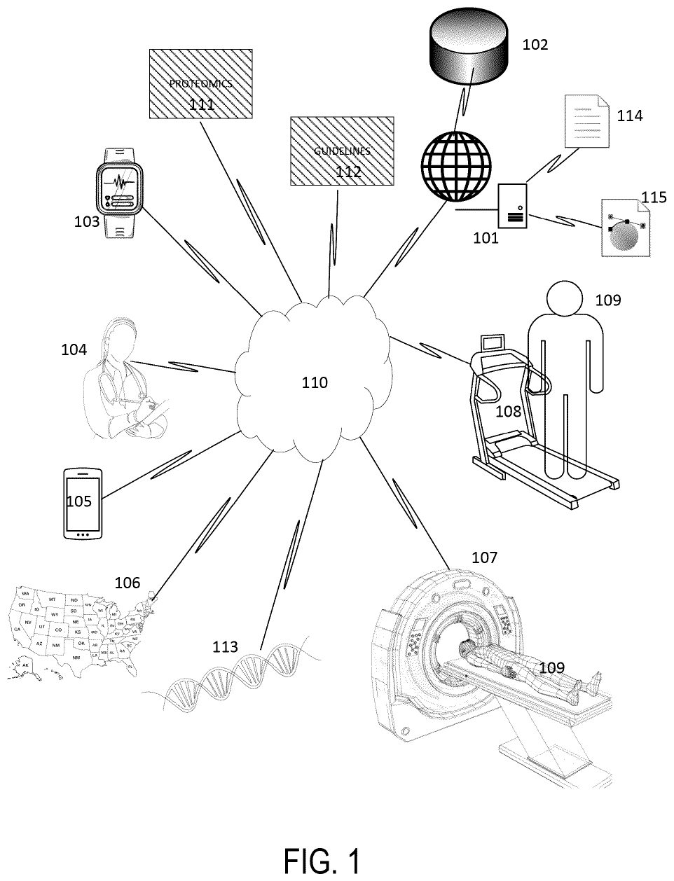

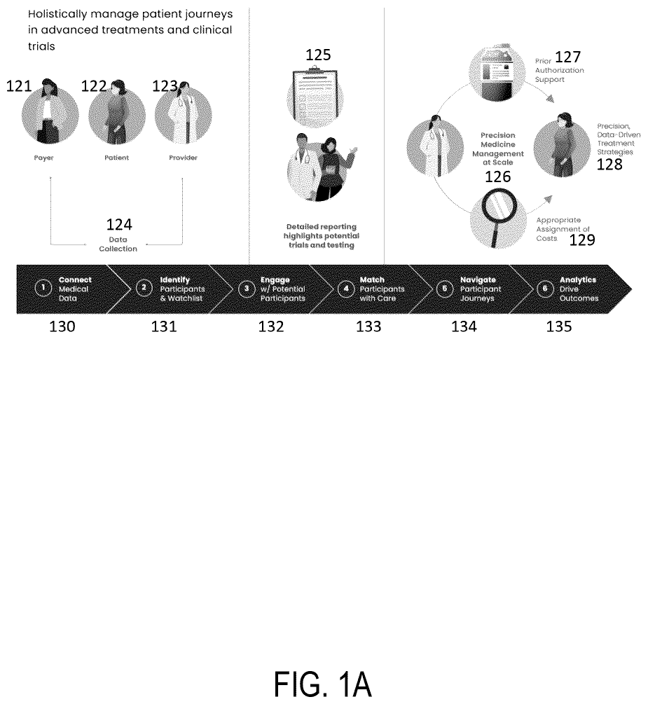

Referring now to FIG. 1 a schematic diagram illustrates sources of disparate data that may be aggregated in a Controller 101 that may include an AI Engine, and stored in a data storage 102.

Sources may include, by way of non-limiting example, one or more of: data from healthcare practitioners 104, data from payers (such as, insurance providers), data from patients 109, data from pharmaceutical developers, data from clinical trial coordinators, and machines that quantify physiological aspects of the patient. By way of non-limiting example, the devices associated with sources may include, one or more of: wearable physiological measurement devices 103; computers and/or smart devices 105, geographic location devices 106, imaging devices 107, physical exertion measuring devices 108, proteomics 111, guidelines 112, genomics 113 and/or a patient 109.

In some embodiments, image data generated by one or more imaging devices 107 may be received into a Controller 101 as objective physiological input that quantifies one or more conditions that relate to patient health conditions. Preferably the image data is time sequenced over a period of time in periodic increments suitable to capture and quantify a progression of a health condition (such as, for example, a disease state). Data may be received via a distributed network 110 or other communications medium.

The controller 101 may analyze the image data in the image data's native form or in a modified form, such as, for example, a user interface 115 including a pattern of one or more of: pixels, polygons and lines that represent the image data in its native form, and/or a textual content 114 descriptive recognized artifacts in the user interface 115. The Controller 101 may recognize certain pixel patterns and/or a progression of pixel patterns. Some embodiments may include the Controller 101 associating the pixel patterns and/or a progression of pixel patterns with a health condition and/or a health diagnosis. In some embodiments, a health diagnosis may be used to relate to a particular clinical trial or set of clinical trials, or to request. A health diagnosis may also be included in a request for participation in a clinical trial, a particular health treatment, or for healthcare insurance reimbursement.

Other embodiments may not require a health diagnosis, in such embodiments, the controller 101 may associate certain patterns, or arrangement of patterns with data sets that indicate those patterns may be successfully treated with via a particular clinical trial and/or set of clinical trials.

A health treatment strategy may additionally be based upon data indicating that participation in one or more particular clinical trials may preclude participation in another clinical trial. In addition, a chronological timeline may set forth overlapping time periods that preclude participation in specific clinical trials with conflicting time periods.

In some embodiments, controller 101 generated treatment strategies may include suggested courses of action that may be weighted based upon one or more of: projected efficacy; timing, geographic location and a patient's ability to be transported; cost; and health condition criticality (including for example whether the FDA may grant a compassion based permission to enter a patient into a trial of a treatment protocol).

Referring now to FIG. 1A, data collection 124 may include collection of data from one or more of: a payer 121, a patient 122, and a provider 123. Data sources 121,122,123 may provide input to automated systems that are operative to holistically managed patient journeys towards advanced treatments and/or clinical trials. Detailed reporting 125 highlights the potential of the trials and the testing. In some embodiments, precision medicine management at scale 126 may include: prior authorization and support 127, precision data driven treatment strategies 128, and appropriate assignment of costs 129.

In a timeline continuum, steps along a participants healthcare journey may include connecting medical data 130 with the identification of participants, and identifying participants and a watch list 131, engaging with potential participants 132, matching participants with care 133, navigating participant journeys 134, and deriving analytics to drive outcomes 135.

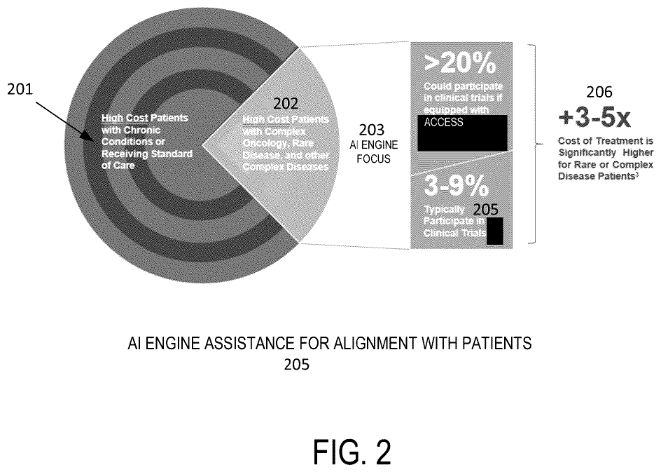

Referring to FIG. 2 a system including AI engine assistance for alignment of patients 205 may include high-cost patients with chronic conditions or patients receiving standards of care 201. To filter out high-cost patients with complex oncology, rare disease, and other complex diseases 202, an AI engine can focus 203, on the high-cost patients with complex oncology 202, to provide lists of patients that can participate in clinical trials if equipped with access 204. Such patients can be greater than 20% of the entire patient pool 2013. Nine percent (9%) of patients typically do participate in clinical trials 207. The cost of treatment is significantly higher for rare or complex disease patients, generally three to five times higher 206.

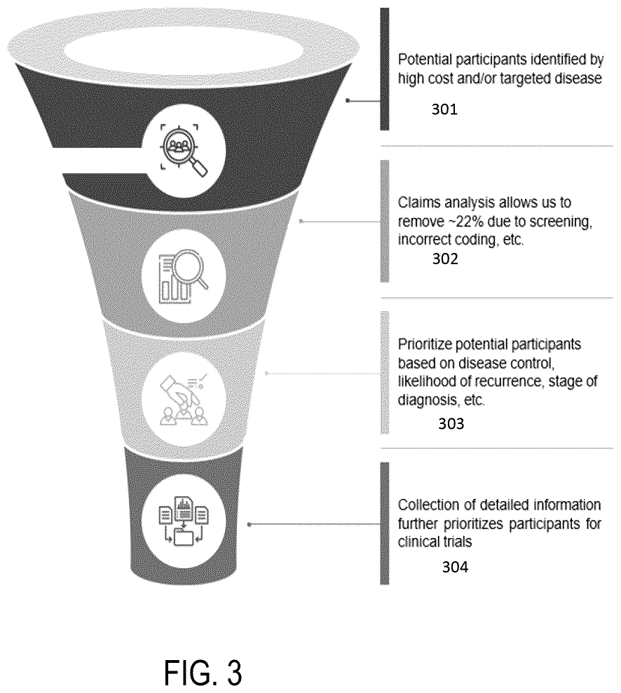

Referring now to FIG. 3 potential participants identified as high-cost and/or targeted disease 301 can input their data, which includes a claims analysis that allows a controller to remove approximately 22% due to screening, incorrect coding, faulty data, or other reasons 302. The controller can prioritize potential participants based on disease control, likelihood of occurrence, stage of diagnosis, or other factors 303, and finally, collection of detailed information further prioritizes participants for clinical trials 304.

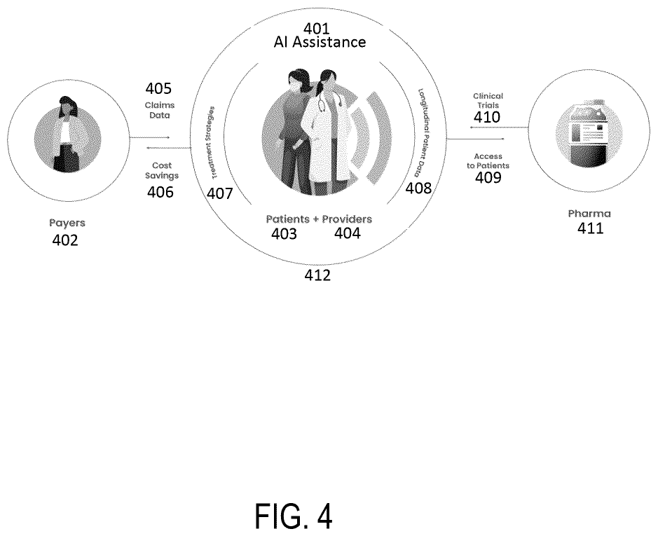

Referring now to FIG. 4, a system that can provide AI assistance 401 can implicate and benefit payers 402 with cost savings 406. The payers may submit claims data 405 into a controller. Patients 403 and providers 404 can also submit data. Treatment strategies 407 are input into the controller 407 and longitudinal patient data 408 can lead to access to patients 409 for clinical trials 410. The beneficiary of the increased access to patients 409, can include the pharmaceutical companies 411.

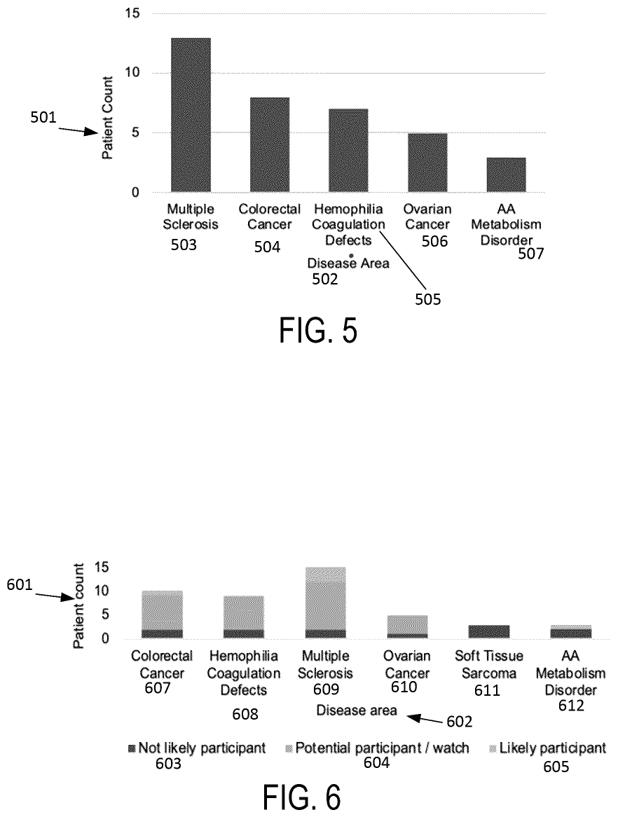

Referring now to FIG. 5, a graph illustrates relationships between patient count 501 and disease areas 502. Disease areas 502 may include one or more of, for example: multiple sclerosis 503, colorectal cancer 504, hemophilia, coagulation defects 505, ovarian cancer 506, and metabolism disorders 507. Other disease areas are also included in the present invention.

Referring now to FIG. 6, a user interface may include an indication of patient count 601 that designates unlikely participants 603, potential participants 604 that are put on watch, and likely participants 605 that are recommended for inclusion in a clinical. Disease states 602 that might be used in the clinicals may include, for example, colorectal cancer 607, hemophilia, coagulation defects 608, multiple sclerosis 609, ovarian cancer 610, soft tissue sarcoma 611, and metabolism disorders 612. Other disease states may also be included.

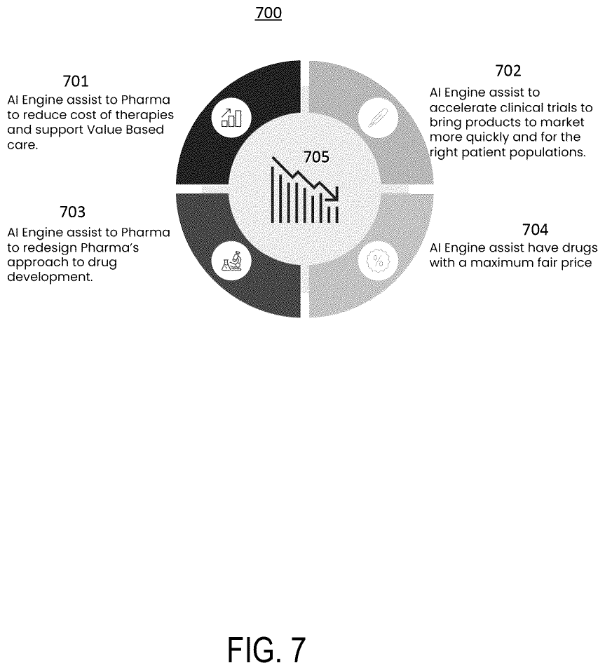

Referring now to FIG. 7, a chart that might be included in the user interface 700 may include AI engine assistance to pharma to reduce cost of therapies and support value-based care 701, AI engine assistance to accelerate clinical trials to bring products to market more quickly and for the right patient populations 702, AI assistance to pharma to redesign pharma approach to drug development 703, and AI assistance to have drugs with a maximum fair price 704.

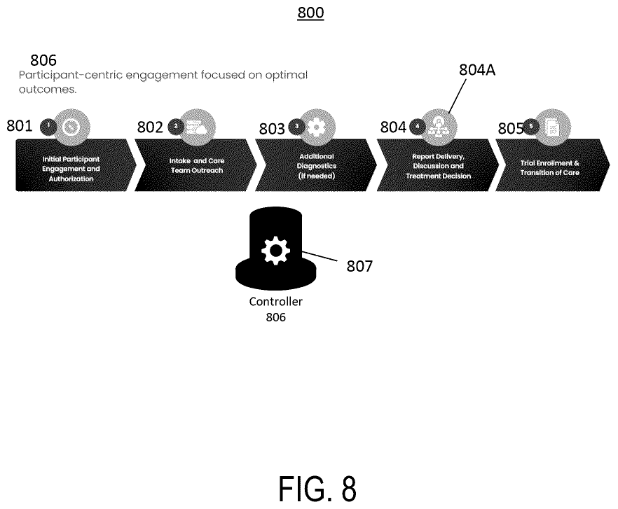

Referring now to FIG. 8, a timeline 800 is provided that shows key points involved in a process for participant-centric engagement, focused on optimal outcomes 806. The timeline 800 may commence with initial participant engagement and authorization is received 801 and input into the controller 806. Intake and care team outreach may be made to the participant 802. Additional diagnostics may be indicated 803, and a report 804A may be generated and delivered that discusses treatment decisions 804. Trial enrollment and transition of care 805 may conclude the timeline.

Initial participant engagement and authorization may include a navigator initiating introductions or a care manager can take and make warm introductions. Appointments may be scheduled, and an invitation may be sent to each participant. HIPAA compliant documents may also be shared for authorization via a controller 806. At Step 802 member and provider orientations are input into the controller. Workflow navigator completes the initial intake and medical history. Signed forms are received and logged, and the navigator reaches out again to the participant's care team. The controller 806, which may include an AI engine 807 reviews the participant's clinical data and history to identify gaps and any additional objective or subjective physiological data, such as diagnostics, which may be useful.

The controller 806 may generate report 804A which may be shared with participants, physicians, clinicians, and clinical overseers. The report may include strategies, and/or outline details to the participant and the physician which are useful to make a go-forward decision. If the participant chooses to pursue the clinical trial, trial enrollment 805 may proceed. The navigator may manage the referral, the enrollment process, and help with adherence and questions throughout the trial. Warm transitions for care are also provided when needed.

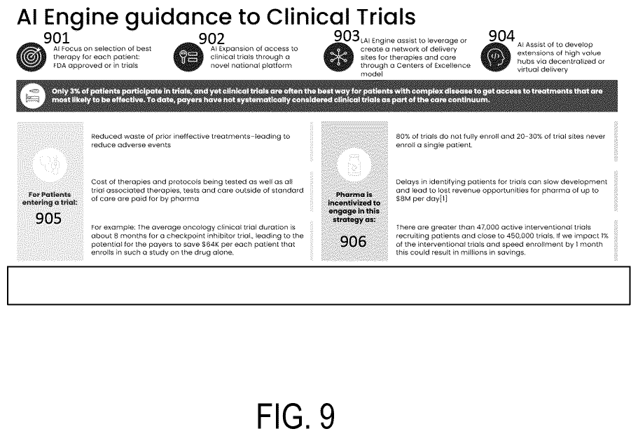

Referring now to FIG. 9, an AI engine can be used for guidance to clinical trials. At Step 901 an initial AI focus can be on the selection of a best therapy for each patient and which trials are FDA approved or in trials.

At Step 902 AI can expand the access to clinical trials through a new national platform. At Step 903 the AI engine may assist to leverage or create a network of delivery sites for therapies and care through a Centers of Excellence model.

At Step 904 the AI may assist in the development of extensions of high value hubs with decentralized or virtual delivery for patients in the trial 905. There is reduced waste of prior ineffective treatments, leading to reduced adverse events, the cost of therapies and protocols being tested, as well as all trial-associated therapies, tests, and care outside of standard of care are paid for by the pharma. For example, an average oncology clinical trial duration is about eight months for a checkpoint inhibitor trial, leading to the potential for the payers to save $64,000 for each patient that enrolls in such a study on the drug alone.

At Step 906 Pharma may be incentivized to engage in this strategy for reasons that may include 80% of trials do not fully enroll, and 20 to 30% of trial sites never enroll a single patient. For example, if there are delays in identifying participants for trials. Development may be slowed and lead to lost revenue opportunities for trial sponsors, which may include costs of millions of dollars per day.

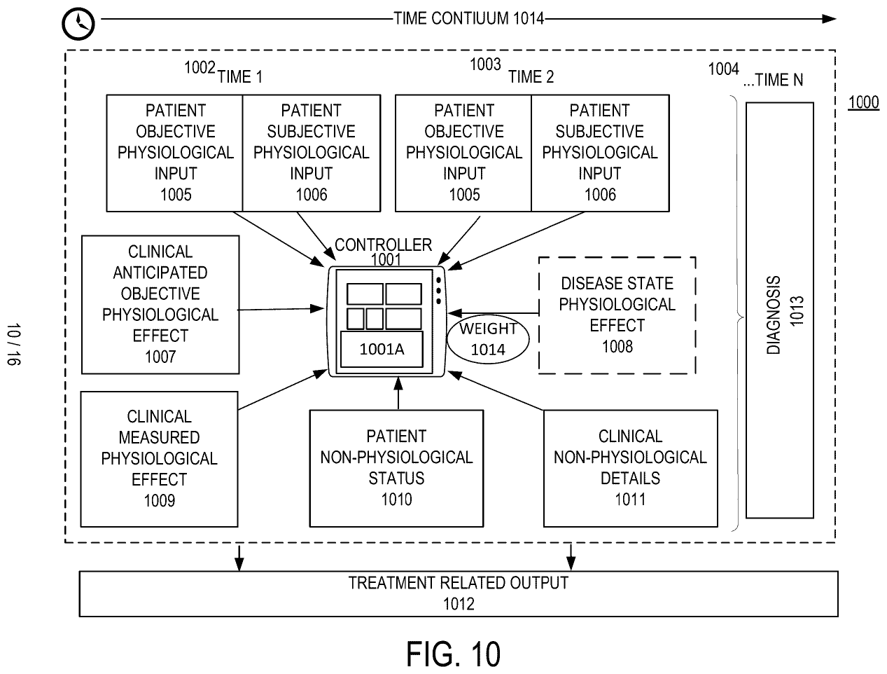

Referring now to FIG. 10 a controller 1001 may receive input from multiple input sources (e.g., 1005-1011) and produce a treatment related output 1012. In preferred embodiments, the controller 1001 will include hardware and software suitable to act as an AI Engine 1001A.

Data input may include, by way of non-limiting example, one or both of: patient objective physiological data 1005 and patient subjective physiological data 1006.

Patient objective physiological data 1005 may include a quantification of a biological state existing within the patient. By way of non-limiting example, patient objective physiological data 1005 may include, one or more of: drugs, genomics, and other biomarkers. clinical trial meta data

In some preferred embodiments, patient objective physiological data 1005 results from a patient's interaction with a device, diagnostic tool, or apparatus and/or observation made by a device, diagnostic tool, or apparatus. Devices, diagnostic tools, and apparatus, may include, by way of non-limiting example, one or more of:
Electrocardiogram (ECG or EKG): to measure electrical activity of the heart.
Blood Pressure Monitor (Sphygmomanometer): to measure arterial blood pressure.
Pulse Oximeter: to measure oxygen saturation in the blood.
Electronic Stethoscope: to measure heart and lung functionality.
Holter Monitor: a portable ECG to monitor heart rhythms over an extended period.
Doppler Ultrasound: to measure blood flow in arteries and veins.
Spirometer: to measure lung capacity and airflow.
Peak Flow Meter: to measure how quickly one can blow out air.
Pulmonary Function Test (PFT) Equipment: to assess how well lungs are functioning.
Capnograph: to measure the concentration of carbon dioxide in exhaled air.
Electroencephalogram (EEG): to measure electrical activity of the brain.
Electromyography (EMG): to measure electrical activity in muscles.
Nerve Conduction Study Equipment: to measure how quickly electrical signals move through a nerve.
Goniometer: to measure joint angles and range of motion.
Dynamometer: to measure force exerted by a muscle.
Myograph: to measure muscle tension.
Glucometer: to measure blood sugar levels.
Continuous Glucose Monitor (CGM): to track blood glucose levels throughout the day and night.
Thermometer: to measure body temperature. Types may include oral, rectal, tympanic (ear), and temporal (forehead) thermometers.
Thermal Imaging Camera: to measure skin surface temperature.
Dermatoscope: to magnify and illuminate the skin to detect any abnormalities.
Patch Test: to identify allergens that may trigger skin reactions.
Tewameter: to measure Transepidermal Water Loss (TEWL) which provides info on skin barrier function.
Chromhidrosis Test: to identify variations of sweat.
pH Monitoring: to measure acidity in a patient's esophagus.

Imaging devices may include, by way of non-limiting example, one or more of:
X-ray Machines
   a. Purpose: Produces images of bones, chest, and certain soft tissues.
   b. Uses: Diagnosing fractures, infections, lung diseases, and tumors.
   c. Types: Standard X-ray, digital X-ray.
Computed Tomography (CT) Scanners
   a. Purpose: Uses X-rays and computer processing to create detailed cross-sectional images of the body.
   b. Uses: Detecting tumors, internal injuries, and abnormalities in the brain, chest, and abdomen.
Magnetic Resonance Imaging (MRI) Machines
   a. Purpose: Uses strong magnetic fields and radio waves to create detailed images of soft tissues.
   b. Uses: Diagnosing neurological conditions, musculoskeletal issues, and internal organ diseases.
   c. Types: Open MRI, closed MRI.
Ultrasound Machines
   a. Purpose: Uses high-frequency sound waves to produce real-time images of internal organs and tissues.
   b. Uses: Pregnancy monitoring, assessing organs like the liver, kidneys, and heart, and guiding biopsies.
Positron Emission Tomography (PET) Scanners
   a. Purpose: Produces 3D images of metabolic activity using a radioactive tracer.
   b. Uses: Detecting cancer, evaluating brain disorders, and assessing heart function.
Single Photon Emission Computed Tomography (SPECT) Scanners
   a. Purpose: Similar to PET but uses gamma rays to visualize blood flow and organ function.
   b. Uses: Heart disease diagnosis, brain imaging, and detecting bone disorders.
Mammography Machines
   a. Purpose: Specialized X-ray machines designed to image breast tissue.
   b. Uses: Screening and diagnosing breast cancer.
Bone Densitometry (DEXA) Scanners
   a. Purpose: Measures bone density using X-rays.
   b. Uses: Diagnosing osteoporosis and assessing fracture risk.
Fluoroscopy Machines
   a. Purpose: Produces real-time moving X-ray images of internal structures.
   b. Uses: Guiding catheter insertions, barium studies, and joint injections.
Endoscopy Systems
   a. Purpose: Combines imaging with a flexible camera inserted into the body.
   b. Uses: Examining the digestive tract, respiratory system, or other internal areas.
Nuclear Medicine Imaging Systems
   a. Purpose: Uses radioactive tracers and gamma cameras to visualize organ function.
   b. Uses: Imaging the thyroid, kidneys, heart, and other organs.
Angiography Systems
   a. Purpose: Specialized imaging for visualizing blood vessels using contrast agents and X-rays.
   b. Uses: Diagnosing blockages, aneurysms, and vascular diseases.
Optical Coherence Tomography (OCT) Scanners
   a. Purpose: Uses light waves to capture detailed images of tissues, especially the retina.
   b. Uses: Eye disease diagnosis, such as glaucoma or macular degeneration.
Echocardiography Machines
   a. Purpose: Ultrasound machines specifically designed for imaging the heart.
   b. Uses: Evaluating heart function, valve diseases, and detecting congenital heart issues.
C-arm Machines
   a. Purpose: Portable X-ray machines with a C-shaped arm for real-time imaging during surgeries.
   b. Uses: Orthopedic, vascular, and cardiac procedures.
Thermography Cameras
   a. Purpose: Detects infrared heat emissions from the body.
   b. Uses: Assessing blood flow, inflammation, or certain pain conditions.
Photoacoustic Imaging Systems
   a. Purpose: Combines ultrasound and laser-induced sound waves to visualize blood vessels and tumors.
   b. Uses: Emerging technology for cancer detection and vascular studies.
Hybrid Imaging Systems
   a. Examples: PET/CT, SPECT/CT, PET/MRI.
   b. Purpose: Combines two imaging modalities to provide both functional and structural data.
   c. Uses: Advanced cancer, heart, and neurological assessments.

Image data may be converted from an input format into one or more raster images comprising patterns of pixels. Each pixel may have a digital value. In some embodiments, a digital value may include, by way of non-limiting example, a binary number with a value of between 0 and 255, or other binary value. Other embodiments may include each pixel being associated with a non-binary number. The controller 1001, which may act as an AI Engine 1001A, may analyze the pixel patterns and correlate patterns derived from patient objective physiological input that includes an image with predicted objective physiological effects 1007 and subjective physiological effects.

Imaging devices may include, by way of non-limiting example, one or more of:
X-ray Machines
   a. Purpose: Produces images of bones, chest, and certain soft tissues.
   b. Uses: Diagnosing fractures, infections, lung diseases, and tumors.
   c. Types: Standard X-ray, digital X-ray.
Computed Tomography (CT) Scanners
   a. Purpose: Uses X-rays and computer processing to create detailed cross-sectional images of the body.
   b. Uses: Detecting tumors, internal injuries, and abnormalities in the brain, chest, and abdomen.
Magnetic Resonance Imaging (MRI) Machines
   a. Purpose: Uses strong magnetic fields and radio waves to create detailed images of soft tissues.
   b. Uses: Diagnosing neurological conditions, musculoskeletal issues, and internal organ diseases.
   c. Types: Open MRI, closed MRI.
Ultrasound Machines
   a. Purpose: Uses high-frequency sound waves to produce real-time images of internal organs and tissues.
   b. Uses: Pregnancy monitoring, assessing organs like the liver, kidneys, and heart, and guiding biopsies.
Positron Emission Tomography (PET) Scanners
   a. Purpose: Produces 3D images of metabolic activity using a radioactive tracer.
   b. Uses: Detecting cancer, evaluating brain disorders, and assessing heart function.
Single Photon Emission Computed Tomography (SPECT) Scanners
   a. Purpose: Similar to PET but uses gamma rays to visualize blood flow and organ function.
   b. Uses: Heart disease diagnosis, brain imaging, and detecting bone disorders.
Mammography Machines
   a. Purpose: Specialized X-ray machines designed to image breast tissue.
   b. Uses: Screening and diagnosing breast cancer.
Bone Densitometry (DEXA) Scanners
   a. Purpose: Measures bone density using X-rays.
   b. Uses: Diagnosing osteoporosis and assessing fracture risk.
Fluoroscopy Machines
   a. Purpose: Produces real-time moving X-ray images of internal structures.
   b. Uses: Guiding catheter insertions, barium studies, and joint injections.
Endoscopy Systems
   a. Purpose: Combines imaging with a flexible camera inserted into the body.
   b. Uses: Examining the digestive tract, respiratory system, or other internal areas.
Nuclear Medicine Imaging Systems
   a. Purpose: Uses radioactive tracers and gamma cameras to visualize organ function.
   b. Uses: Imaging the thyroid, kidneys, heart, and other organs.
Angiography Systems
   a. Purpose: Specialized imaging for visualizing blood vessels using contrast agents and X-rays.
   b. Uses: Diagnosing blockages, aneurysms, and vascular diseases.
Optical Coherence Tomography (OCT) Scanners
   a. Purpose: Uses light waves to capture detailed images of tissues, especially the retina.
   b. Uses: Eye disease diagnosis, such as glaucoma or macular degeneration.
Echocardiography Machines
   a. Purpose: Ultrasound machines specifically designed for imaging the heart.
   b. Uses: Evaluating heart function, valve diseases, and detecting congenital heart issues.
C-arm Machines
   a. Purpose: Portable X-ray machines with a C-shaped arm for real-time imaging during surgeries.
   b. Uses: Orthopedic, vascular, and cardiac procedures.
Thermography Cameras
   a. Purpose: Detects infrared heat emissions from the body.
   b. Uses: Assessing blood flow, inflammation, or certain pain conditions.
Photoacoustic Imaging Systems
   a. Purpose: Combines ultrasound and laser-induced sound waves to visualize blood vessels and tumors.
   b. Uses: Emerging technology for cancer detection and vascular studies.
Hybrid Imaging Systems
   a. Examples: PET/CT, SPECT/CT, PET/MRI.
   b. Purpose: Combines two imaging modalities to provide both functional and structural data.
   c. Uses: Advanced cancer, heart, and neurological assessments.

In some preferred embodiments, one or both of: objective physiological data 1005, and subjective physiological data 1006 are received as input into the controller at different time instance in time, such as a time one 1002 and time two 1003 up and so on up until a time “N” 1004. Time N 1004 may be determined based upon a requisite number of data points to make an accurate conclusion, or be determined upon an available timeframe determined by one or more of: a health state of additional patient, timing of a clinical trial, access to data generating sources (e.g. providers of objective physiological data 1005 and/or subjective physiological data 1006). In this manner, patient related objective physiological data 1005 and patient related subjective physiological data 1006 may be input along a time continuum 1014.

In some embodiments, the controller 1001 may also receive clinical anticipated objective physiological effects 1007 and disease state physiological effects 1008. Preferred embodiments will also have clinical measured subjective physiological effect 1009 and patient non-physiological status 1010 may also be input into the controller 1001. In some preferred embodiments, clinical measured physiological effect 1009 may include clinical trial meta data.

Patient non-physiological status 1010 may include, for example, data descriptive of a patient and/or descriptive of a patient's situation, such as, by way of non-limiting example, one or more of: a patient's age; a patient's geographic location; a patient's ability to travel; special needs of a patient, insurance coverage details; past treatments received by a patient; or other demographic information.

Another modality of data may include clinical non-physiological details, such as for example, venues used to the clinical trial, length of time for a treatment session, anticipated number of treatment sessions, date and times of treatment sessions, or other datum that is not related to a physiological effect.

The controller may process any and/or all input data and provide a treatment related output 1012. In some embodiments, the treatment related output may include information that is useful to determine a likelihood of particular person being suitable as a participant in a particular clinical trial.

In another aspect, in some embodiments, the controller may provide output that designates one or more diagnosis 1013. A controller 1001 may include an AI Engine 1001A that assists in providing one or both of the treatment related output 1012 and the diagnosis 1013.

In some preferred embodiments, the treatment related output 1012 will be generated using one or more of: AI processes, Boolean logic algorithms, and statistical analysis. The treatment related output 1012 may be agnostic to a diagnosis 1013.

A treatment related output that is agnostic to a diagnosis 1013 may be based upon a calculated analysis of success of a particular treatment for a particular participant based upon one or more of: patient objective physiological input 1005, patient subjective physiological input 1006, clinical anticipated objective physiological effect 1007, clinical measured physiological effect 1009 patient non-physiological status 1010, clinical non-physiological details 1011, and disease state physiological effect 1008. The treatment related output 1012 may be data and algorithm driven and does not need to be associated with a diagnosis 1013 since a diagnosis may be distracting and may not add to the likelihood of success of a particular clinical trial and/or treatment protocol.

Notwithstanding the foregoing, if a diagnosis is desired, the controller 1001 and in particular an AI Engine 1001A running on the controller may significantly aid in a conclusion of a health state quantified as a diagnosis 1013. The diagnosis 1013 may be useful, for example, during medical insurance transactions and approved uses of controlled substances and/or devices.

Modalities of data manipulation conducted by the controller 1001 and/or the AI Engine 1001A, may include, but are not limited to:

Machine Learning (ML): A subset of AI where systems learn from data. Instead of being explicitly programmed, they adjust their operations to optimize for a certain outcome based on the input they receive.

Deep Learning: A subfield of ML using neural networks with many layers (hence “deep”) to analyze various factors of data, such as, for example convolutional neural networks (CNNs) used in image recognition. For example, convolutional neural networks may receive as input image data from scans of various types and generate pixel patterns representative of the scans. The pixel patterns may be compared to a library of other pixel patterns and/or manipulated to emulate progression of a disease state and/or a treatment protocol over time. Successful treatments based upon Deep Learning patterns may be identified and included in a proposed healthcare strategy based upon the Deep Learning findings without ever having to associate the pixel patterns with a particular disease state (which may be left up to the healthcare practitioner).

Natural Language Processing (NLP): Allows systems to understand, interpret, and generate human language. NLP may provide interpretations of voice data. Voice data may be made accessible for example, via recording made during patient examination; triage; during a procedure, post procedure, during patient transport. NLP may be used to access the apparatus described herein during patient diagnosis while a healthcare practitioner is physically involved in administering to a patient.

Robotics: Robots may operate using AI principles, enabling the robots to perform tasks in accurate, specific, and consistent ways. Robots may also be utilized during data collection, such as during scans, physiological measurement acquisition, biometric measurement acquisition and the like.

Knowledge Representation: The methods and apparatus taught herein may receive data in a native or enhanced state and manipulate and transform the received data into a machine learning understandable form (as discussed further below).

Reasoning: The methods and apparatus taught herein may solve deploy logical deduction via expert systems and the like to facilitate decision-making.

Perception: The methods and apparatus taught herein may use algorithms and complex relational processes that allow machines to interpret disparate data sets, including image data, sound data, and alphanumeric data.

Apparatus and methods may be arranged to form one or more of: Neural Networks; Genetic Algorithms; Expert Systems; and Reinforcement Learning.

In some embodiments, GPUs may be used to accomplish large-scale machine learning models using parallel processing capabilities. Hardware accelerators may be utilized for deep learning tasks. In some embodiments, tensor processing units and/or neuromorphic computing mechanisms may be used to analyze data sets. Cloud platforms may be used with AI processes, such as deep learning that require significant computational resources.

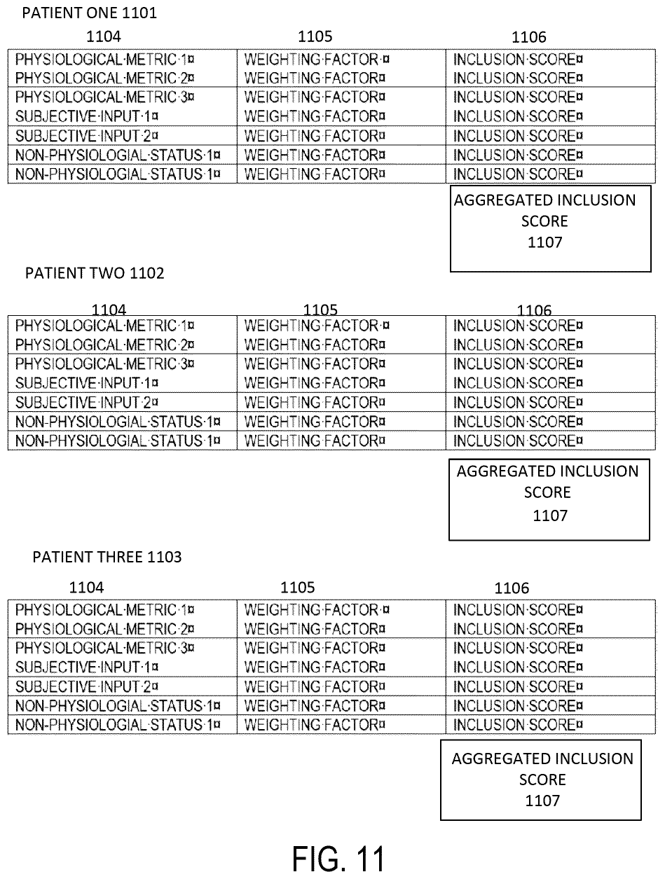

Referring now to FIG. 11, weighting factors 1105 may be associated with one or more patient specific attributes 1104 to assist automated decision by a controller and/or an AI Engine, making a determination as to whether a particular patient 1101-1103 is a good candidate to be a participant in a clinical trial. Non-limiting examples of patient specific attributes may include one or more of the following: physiological metrics, subjective input, and non-physiological status. A weighting factor 1105 may be applied to each metric, or one or more specific metrics to generate an inclusion score 1106. Inclusion scores may be aggregated to generate an aggregated inclusion score.

The aggregated inclusion score may provide guidance as to whether a particular patient will make a good clinical trial participant.

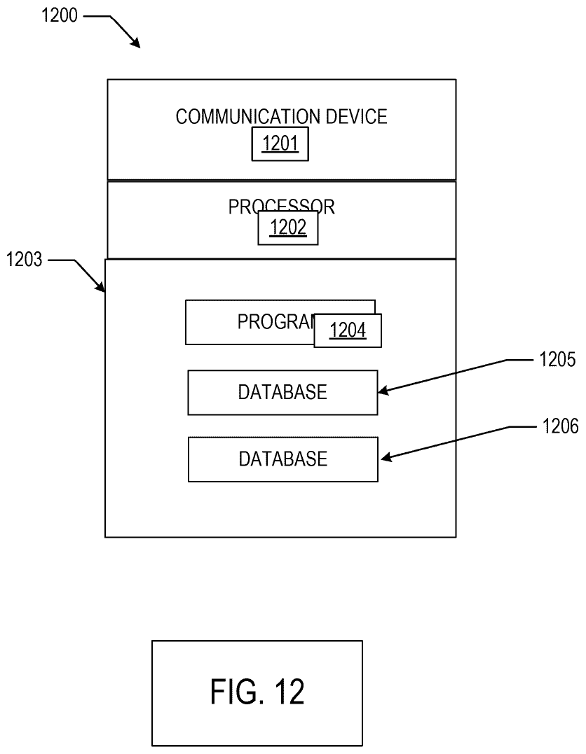

Referring now to FIG. 12, an automated controller is illustrated that may be used to implement various aspects of the present invention in various embodiments, and for various aspects of the present invention. Controller 1200 may be included in one or more of: a wireless tablet or handheld smart device, a server, an integrated circuit incorporated into a Node, appliance, equipment item, machinery, or other automation. The controller 1200 includes a processor unit 1202, such as one or more semiconductor-based processors, coupled to a communication device 1201 configured to communicate via a communication network (not shown in FIG. 12A). The communication device 1201 may be used to communicate, for example, with one or more online devices, such as a smart device, a Node, personal computer, laptop, or a handheld device.

The processor unit 1202 is also in communication with a storage device 1203. The storage device 1203 may comprise any appropriate information storage device, including combinations of digital storage devices (e.g., an SSD), optical storage devices, and/or semiconductor memory devices such as Random Access Memory (RAM) devices and Read Only Memory (ROM) devices.

The storage device 1203 can store a software program 1204 with executable logic for controlling the processor unit 1202. The software program may be executable upon command, such as, via a user input device. The processor unit 1202 performs instructions of the software program 1204, and thereby operates in accordance with the present invention. The processor unit 1202 may also cause the communication device 1201 to transmit information, including, in some instances, timing transmissions, digital data and control commands to operate apparatus to implement the processes described above. The storage device 1203 can additionally store related data in a database 1205 and database 1206, as needed.

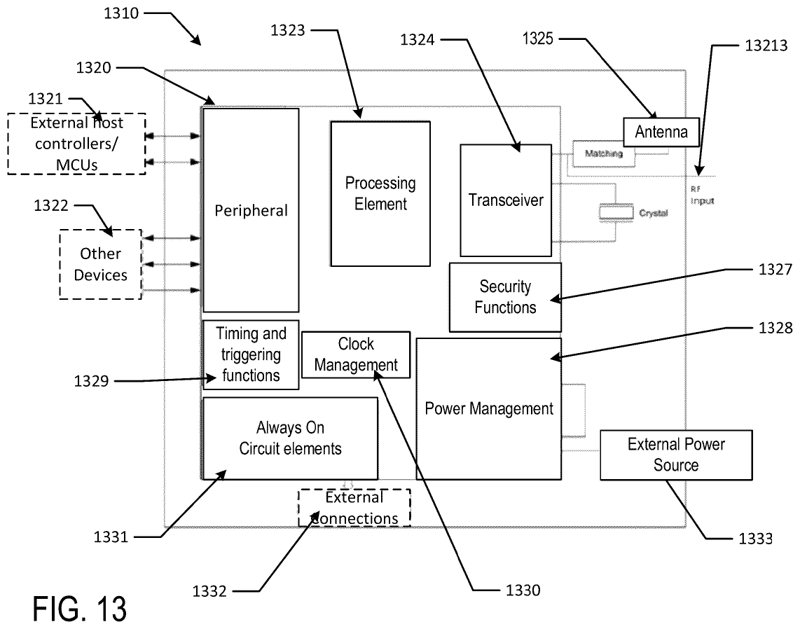

Referring now to FIG. 13, a block diagram of an exemplary controller 1302 is illustrated.

The controller 1302 comprises logic 1326 to interact with the various other components, possibly processing the received signals into different formats and/or interpretations. Logic 726 may be operable to read and write data and program instructions stored in associated storage or memory 1330 such as RAM, ROM, flash, or other suitable memory. It may read a time signal from the clock unit 1328. In some embodiments, the controller 1302 may have an on-board power supply 1332. In other embodiments, the controller 1302 may be powered from a tethered connection to another device, such as a Universal Serial Bus (USB) connection.

The controller 1302 also includes a network interface 1316 to communicate data to a network and/or an associated computing device. Network interface 1316 may provide two-way data communication. For example, network interface 1316 may operate according to the internet protocol. As another example, network interface 1316 may be a local area network (LAN) card allowing a data communication connection to a compatible LAN. As another example, network interface 1316 may be a cellular antenna and associated circuitry which may allow the mobile device to communicate over standard wireless data communication networks. In some implementations, network interface 1316 may include a Universal Serial Bus (USB) to supply power or transmit data. In some embodiments other wireless links may also be implemented.

In some embodiments, the controller 1302 includes an optical capture device 1308 to capture an image and convert it to machine-compatible data. An optical path 1306, typically a lens, an aperture, or an image conduit to convey the image from the rendered document to the optical capture device 1308. The optical capture device 1308 may incorporate a CCD, a Complementary Metal Oxide Semiconductor (CMOS) imaging device, or an optical Sensor 724 of another type.

A microphone 1310 and associated circuitry may convert a sound of an environment, such as spoken words, heartbeats, or other audible data, into machine-compatible signals. A user interface may include input facilities may include, but are not limited to buttons, scroll wheels, or other tactile Sensors such as touch screens, keyboards, and the like.

Visual feedback to the user is possible through a visual display, touchscreen display, or indicator lights. Audible feedback 1334 may come from a loudspeaker or other audio transducer. Tactile feedback may come from a vibrate module 1336.

A motion Sensor 1338 and associated circuitry convert the motion of the controller 1302 into machine-compatible signals. The motion sensor 1338 may comprise an accelerometer that may be used to sense measurable physical acceleration, orientation, vibration, and other movements. In some embodiments, motion sensor 1338 may include a gyroscope or other device to sense different motions.

A location Sensor 1340 and associated circuitry may be used to determine the location of the device. The location Sensor 1340 may detect Global Position System (GPS) radio signals from satellites or may also use assisted GPS where the mobile device may use a cellular network to decrease the time necessary to determine location. In some embodiments, the location Sensor 740 may use radio waves to determine the distance from known radio sources such as cellular towers to determine the location of the controller 1302. In some embodiments these radio signals may be used in addition to GPS.

As an example of one use of controller 1302, a reader may scan some coded information from a location marker in a Structure with the controller 1302. The coded information may be included on apparatus such as a hash code, bar code, RFID, or other data storage device. In some embodiments, the scan may include a bit-mapped image via the optical capture device 1308. Logic 1326 causes the bit-mapped image to be stored in memory 1330 with an associated time-stamp read from the clock unit 1328. Logic 1326 may also perform optical character recognition (OCR) or other post-scan processing on the bit-mapped image to convert it to text. Logic 1326 may optionally extract a signature from the image, for example by performing a convolution-like process to locate repeating occurrences of characters, symbols, or objects, and determine the distance or number of other characters, symbols, or objects between these repeated elements. The reader may then upload the bit-mapped image (or text, or other signature, if post-scan processing has been performed by Logic 1326) to an associated computer via network interface 1316.

As an example of another use of controller 1302, a reader may capture some text from an article as an audio file by using microphone 1310 as an acoustic capture port. Logic 1326 causes audio file to be stored in memory 1330. Logic 1326 may also perform voice recognition or other post-scan processing on the audio file to convert it to text. As above, the reader may then upload the audio file (or text produced by post-scan processing performed by logic 1326) to an associated computer via network interface 1316.

A directional Sensor 1341 may also be incorporated into the controller 1302. The directional Sensor may be a compass and may produce data based upon a magnetic reading or based upon network settings.

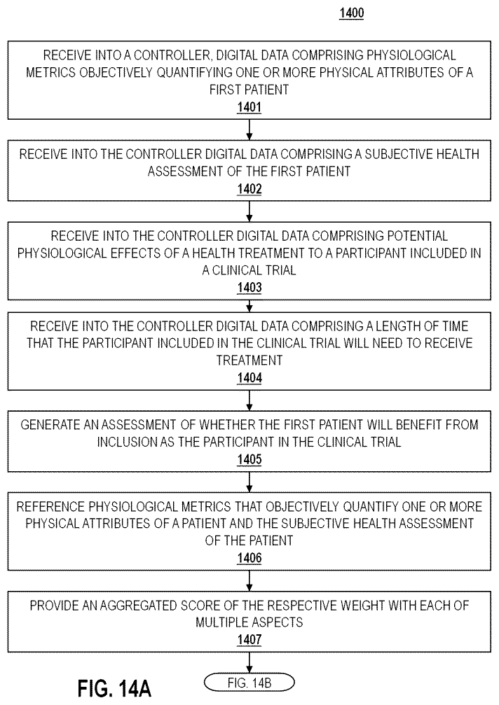

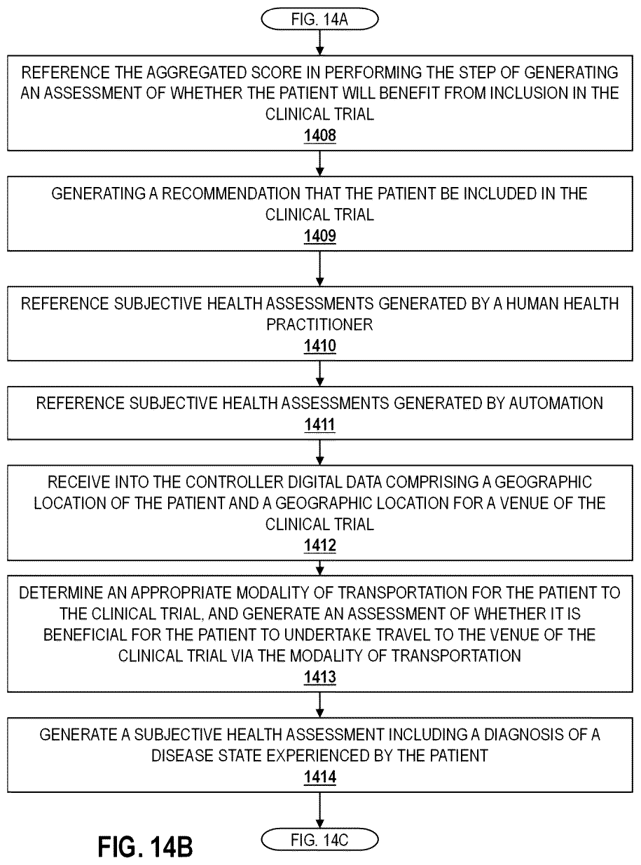

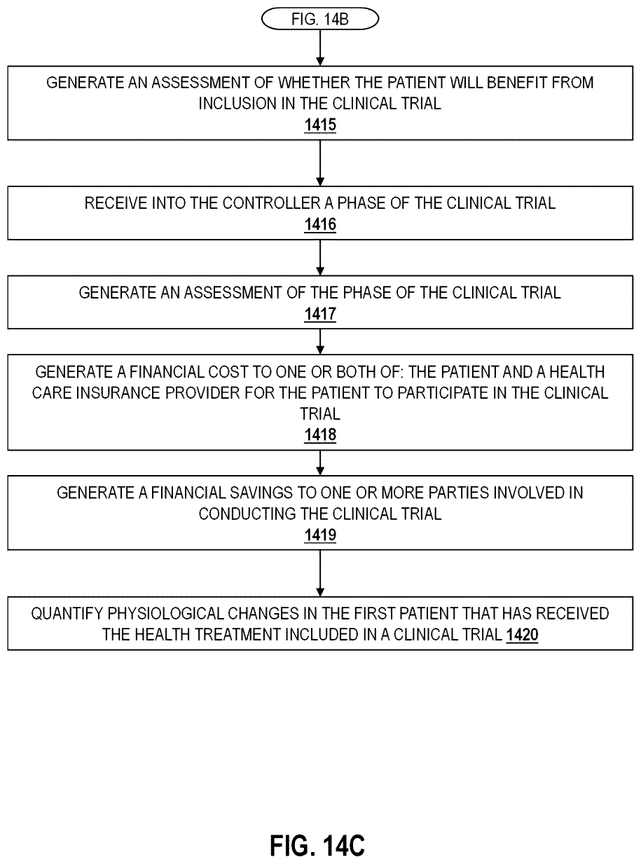

Referring now to FIGS. 14A-14C, flowcharts include method steps that may be executed according to the present invention. The steps include, by way of non-limiting example, one or more of the following steps, in any order conducive to a desired result:

At step 1401, receiving into a controller, digital data comprising physiological metrics objectively quantifying one or more physical attributes of a first patient;

At step 1402, receiving into the controller digital data comprising a subjective health assessment of the first patient;

At step 1403, receiving into the controller digital data comprising potential physiological effects of a health treatment to a participant included in a clinical trial;

At step 1404, receiving into the controller digital data comprising a length of time that the participant included in the clinical trial will need to receive treatment; and

At step 1405, generating an assessment of whether the first patient will benefit from inclusion as the participant in the clinical trial.

At step 1407, providing an aggregated score or the respective weight of each of multiple aspects including: subjective health assessment of the first patient; potential physiological effects of a health treatment to a participant included in a clinical trial; objective health assessment of the first patient; length of time that the participant included in the clinical trial will need to receive treatment; whether the first patient will benefit from inclusion as the participant in the clinical trial; pr any of the other data types included in this disclosure or that may be beneficial to a determination of whether a particular patient is a good candidate for inclusion in a clinical trial or specialized treatment.

At step 1408, referencing the aggregated score in performing the step of generating an assessment of whether the patient will benefit from inclusion in the clinical trial. The step of generating the assessment of whether the patient will benefit from inclusion in the clinical trial may be accomplished by associating a respective weight with each of multiple aspects included in one or both of: the physiological metrics objectively quantifying one or more physical attributes of a patient and the subjective health assessment of the patient.

At step 1409, generating a recommendation that the patient be included in the clinical trial.

At step 1410, referencing a subjective health assessment generated by a human health practitioner.

At step 1411, referencing a subjective health assessment generated by automation.

At step 1412, receiving into the controller digital data comprising a geographic location of the patient and a geographic location for a venue of the clinical trial;

At step 1413, determining an appropriate modality of transportation for the patient to the clinical trial, and generating an assessment of whether it is beneficial for the patient to undertake travel to the venue of the clinical trial via the modality of transportation.

At step 1414, generating a subjective health assessment comprises a diagnosis of a disease state being experienced by the first patient.

At step 1415, generating an assessment of whether the patient will benefit from inclusion in the clinical trial.

At step 1416, receiving into the controller a phase of the clinical trial.

At step 1417, generating an assessment of the phase of the clinical trial.

At step 1418, generating a financial cost to one or both of: the patient and a health care insurance provider for the patient to participate in the clinical trial.

At step 1419, generating a financial savings to one or more parties involved in conducting the clinical trial.

At step 1420, quantifying physiological changes in the first patient that has received the health treatment included in a clinical trial.

While the invention has been described in conjunction with specific embodiments, it is evident that many alternatives, modifications, and variations will be apparent to those skilled in the art in light of the foregoing description. Accordingly, this description is intended to embrace all such alternatives, modifications and variations as fall within its spirit and scope.

What is claimed is:

1. Apparatus for automated improved matching of patients with clinical trials, the apparatus comprising:
a. a controller comprising a processor and a digital storage;
b. a communications device in logical communication with the controller and capable of transceiving digital data;
c. executable software stored on the digital storage and executable upon command to cause the controller to perform the following steps:
i. receive in digital data comprising physiological metrics objectively quantifying one or more physical attributes of a first patient;
ii. receive in digital data comprising a subjective health assessment of the first patient;
iii. receive in digital data comprising potential physiological effects of a health treatment to a participant included in a clinical trial;
iv. receive in digital data comprising a length of time that the participant included in the clinical trial will need to receive treatment; and
v. generate an assessment of whether the first patient will benefit from inclusion as the participant in the clinical trial.

2. The apparatus of claim 1, wherein the step of generating the assessment of whether the first patient will benefit from inclusion as the participant in the clinical trial comprises additional steps of: associating a respective weight with each of multiple aspects included in one or both of: the physiological metrics objectively quantifying one or more physical attributes of a patient and the subjective health assessment of the patient; providing an aggregated score of the respective weight with each of the multiple aspects; and referencing the aggregated score in performing the step of generating the assessment of whether the patient will benefit from inclusion as the participant in the clinical trial.

3. The apparatus of claim 1, wherein the software is executable upon command to additionally cause the controller to: generate a recommendation that the first patient be included in the clinical trial.

4. The apparatus of claim 1, wherein the subjective health assessment is generated by a human health practitioner.

5. The apparatus of claim 1, wherein the subjective health assessment is generated by automation.

6. The apparatus of claim 1, wherein the software is executable upon command to additionally cause the controller to: receive in digital data comprising a geographic location of the first patient and a geographic location for a venue of the clinical trial; determine an appropriate modality of transportation for the first patient to the clinical trial, and generate an assessment of whether it is beneficial for the first patient to undertake travel to the venue of the clinical trial via the appropriate modality of transportation.

7. The apparatus of claim 1, wherein the subjective health assessment comprises a diagnosis of a disease state being experienced by the first patient.

8. The apparatus of claim 1, wherein the software is executable upon command to additionally cause the controller to generate the assessment of whether the first patient will benefit from inclusion as the participant in the clinical trial.

9. The apparatus of claim 1, wherein the software is executable upon command to additionally cause the controller to receive into the controller a phase of the clinical trial.

10. The apparatus of claim 9, wherein the software is executable upon command to additionally cause the controller to generate an assessment of the phase of the clinical trial.

11. The apparatus of claim 1, wherein the software is executable upon command to additionally cause the controller to generate a financial cost to one or both of: the first patient and a health care insurance provider for the first patient to participate in the clinical trial.

12. The apparatus of claim 1, wherein the software is executable upon command to additionally cause the controller to generate a financial savings to one or more parties involved in conducting the clinical trial.

13. The apparatus of claim 12, wherein the one or more parties involved in conducting the clinical trial comprise one or more of: a sponsor, a principal investigator; a clinical research team, the participant, and an institutional review board.

14. The apparatus of claim 1, wherein the software is executable upon command to additionally cause the controller to receive data quantifying physiological changes in the first patient that has received the health treatment included in the clinical trial.

15. The apparatus of claim 14, wherein the software is executable upon command to additionally cause the controller to reference the data quantifying physiological changes in the first patient that has received the health treatment included in the clinical trial, and generate an assessment of whether the first patient has benefited from receiving the health treatment included in the clinical trial.

16. The apparatus of claim 14, wherein the software is executable upon command to additionally cause the controller to repeat steps 1. to v. for a second patient and referencing the data quantifying physiological changes in the first patient to generate the assessment of whether the second patient will benefit from inclusion in the clinical trial.

17. A method for automated improved matching of patients with clinical trials, the method comprising:
a. receiving into a controller, digital data comprising physiological metrics objectively quantifying one or more physical attributes of a first patient;
b. receiving into the controller, digital data comprising a subjective health assessment of the first patient;
c. receiving into the controller, digital data comprising potential physiological effects of a health treatment to a participant included in a clinical trial;
d. receiving into the controller, digital data comprising a length of time that the participant included in the clinical trial will need to receive treatment; and
e. generating an assessment of whether the first patient will benefit from inclusion as the participant in the clinical trial.

18. The method of claim 17, further comprising the step of associating a respective weight with each of multiple aspects included in one or both of: the physiological metrics objectively quantifying one or more physical attributes of a patient and the subjective health assessment of the patient; providing an aggregated score of the respective weight with each of multiple aspects; and referencing the aggregated score in performing the step of generating an assessment of whether the patient will benefit from inclusion in the clinical trial.

19. The method of claim 18, further comprising the step of generating a recommendation that the patient be included in the clinical trial.

20. The method of claim 17, wherein the subjective health assessment is generated by a human health practitioner.

21. The method of claim 17, wherein the subjective health assessment is generated by automation.

22. The method of claim 17, further comprising the steps of receiving into the controller, digital data comprising a geographic location of a patient and a geographic location for a venue of the clinical trial; determining an appropriate modality of transportation for the patient to the clinical trial, and generating an assessment of whether it is beneficial for the patient to undertake travel to the venue of the clinical trial via the modality of transportation.

23. The method of claim 17, wherein the subjective health assessment comprises a diagnosis of a disease state being experienced by the first patient.

24. The method of claim 17, further comprising the step of generating an assessment of whether a patient will benefit from inclusion in the clinical trial.

25. The method of claim 17, further comprising the step of receiving into the controller a phase of the clinical trial.

26. The method of claim 17, further comprising the step of generating an assessment of the phase of the clinical trial.

27. The method of claim 17, further comprising the step of generating a financial cost to one or both of: a patient and a health care insurance provider for the patient to participate in the clinical trial.

28. The method of claim 17, further comprising the step of generating a financial savings to one or more parties involved in conducting the clinical trial.

29. The method of claim 28, wherein the parties involved in conducting the clinical trial comprise one or more of: a sponsor, a principal investigator, a clinical research team, the participant, and an institutional review board.

30. The method of claim 17, further comprising the step of receiving data quantifying physiological changes in the first patient that has received the health treatment included in the clinical trial.

31. The method of claim 30, further comprising the step of referencing the data quantifying physiological changes in the first patient that has received the health treatment included in the clinical trial, and generating an assessment of whether the first patient has benefited from receiving the health treatment included in the clinical trial.

32. The method of claim 31, further comprising the step of repeating steps a. to e. for a second patient and referencing the data quantifying physiological changes in the first patient to generate the assessment of whether the second patient will benefit from inclusion in the clinical trial.

33. A method for automated improved matching of patients with clinical trials, the method comprising:
a. receiving into a controller, digital data comprising physiological metrics objectively quantifying one or more physical attributes of multiple patients;
b. receiving into the controller, digital data comprising a subjective health assessment of the multiple patients;
c. receiving into the controller, digital data comprising potential physiological effects of a health treatment included in a clinical trial;
d. receiving into the controller, digital data comprising a length of time that each participant in the clinical trial will need to receive treatment; and
e. generating a list of candidates, chosen from the multiple patients who will benefit from inclusion in the clinical trial.

34. The method of claim 33, further comprising the step of generating an assessment of whether each candidate will benefit from inclusion in the clinical trial.

35. The method of claim 33, further comprising the step of generating a recommendation that each candidate be included in the clinical trial.

36. The method of claim 33, further comprising the step of generating a financial cost to one or both of: each candidate and a health care insurance provider for each candidate to participate in the clinical trial.
